SEQUENCE LISTING (27 sequences)

SEQ ID NO: 1 - protein, 44 aa, Artificial Sequence
  Synthetic Peptide
HGEGTFTSDLSKQMEEEAVRLFIEWLKNGGPSSGAPPSKKKKKK

SEQ ID NO: 2 - protein, 36 aa, Artificial Sequence
  Synthetic Peptide
HGEGTFTSDLSKQMEEEAVRLFIEWLKNGGPSSGAS

SEQ ID NO: 3 - protein, 42 aa, Artificial Sequence
  Synthetic Peptide
HGEGTFTSDLSKQMEEEAVRLFIEWLKNGGPSSGASKKKKKK

SEQ ID NO: 4 - protein, 50 aa, Artificial Sequence
  Synthetic Peptide
KKKKKKHGEGTFTSDLSKQMEEEAVRLFIEWLKNGGPSSGAPPSKKKKKK

SEQ ID NO: 5 - protein, 42 aa, Artificial Sequence
  Synthetic Peptide
KKKKKKHGEGTFTSDLSKQMEEEAVRLFIEWLKNGGPSSGAS

SEQ ID NO: 6 - protein, 48 aa, Artificial Sequence
  Synthetic Peptide
KKKKKKHGEGTFTSDLSKQMEEEAVRLFIEWLKNGGPSSGASKKKKKK

SEQ ID NO: 7 - protein, 50 aa, Artificial Sequence
  Synthetic Peptide
NEEEEEHGEGTFTSDLSKQMEEEAVRLFIEWLKNGGPSSGAPPSKKKKKK

SEQ ID NO: 8 - protein, 42 aa, Artificial Sequence
  Synthetic Peptide
NEEEEEHGEGTFTSDLSKQMEEEAVRLFIEWLKNGGPSSGAS

SEQ ID NO: 9 - protein, 48 aa, Artificial Sequence
  Synthetic Peptide
NEEEEEHGEGTFTSDLSKQMEEEAVRLFIEWLKNGGPSSGASKKKKKK

SEQ ID NO: 10 - protein, 31 aa, Artificial Sequence
  Synthetic Peptide
HAEGTFTSDVSSYLEGQAAXEFIAWLVRGRG

SEQ ID NO: 11 - protein, 30 aa, Homo Sapiens
HAEGTFTSDVSSYLEGQAAKEFIAWLVKGR

SEQ ID NO: 12 - protein, 31 aa, Homo Sapiens
HAEGTFTSDVSSYLEGQAAKEFIAWLVKGRG

SEQ ID NO: 13 - protein, 31 aa, Homo Sapiens
HSQGTFTSDYAKYLDARRAKEFIAWLVKCRG

SEQ ID NO: 14 - protein, 31 aa, Homo Sapiens
HSQGTFTSDYAKYLDARRAKEFIAWLVKGCG

SEQ ID NO: 15 - protein, 30 aa, Homo Sapiens
  MISC_FEATURE (12)..(12): Xaa is selected from a group consisting of Lys, Arg, Ala
HSQGTFTSDYAXYLDAXXAXEFIXWLVXGR

SEQ ID NO: 16 - protein, 31 aa, Homo Sapiens
  MISC_FEATURE (12)..(12): Xaa is selected from a group consisting of Lys, Arg, Ala
HSQGTFTSDYAXYLDAXXAXEFIXWLVXGRG

SEQ ID NO: 17 - protein, 31 aa, Homo Sapiens
  MISC_FEATURE (12)..(12): Xaa is selected from a group consisting of Lys, Arg, Ala
HSQGTFTSDYAXYLDAXXAXEFIXWLVXGRC

SEQ ID NO: 18 - protein, 32 aa, Homo Sapiens
  MISC_FEATURE (12)..(12): Xaa is selected from a group consisting of Lys, Arg, Ala
HSQGTFTSDYAXYLDAXXAXEFIXWLVXGRGK

SEQ ID NO: 19 - protein, 31 aa, Artificial Sequence
  Synthetic Peptide
HXEGXXTSDXSSYLEXXXAXXFIXWLXXXXX

SEQ ID NO: 20 - protein, 32 aa, Artificial Sequence
  Synthetic Peptide
HXEGXXTSDXSSYLEXXXAXXFIXWLXXXXGP

SEQ ID NO: 21 - protein, 39 aa, Artificial Sequence
  Synthetic Peptide
HXXGXFTXDXXXXXXXXXXXXFIXXXXXXXXXXXXXXXX

SEQ ID NO: 22 - protein, 30 aa, Artificial Sequence
  Synthetic Peptide
XHXEGTFTSDVSSYXEGQAAKXFIXWLKNX

SEQ ID NO: 23 - protein, 30 aa, Artificial Sequence
  Synthetic Peptide
XHXEGTFTSDVSSYXEGQAAKXFIXWLVKX

SEQ ID NO: 24 - protein, 29 aa, Artificial Sequence
  Synthetic Peptide
XHXEGTFTSDVSSYXEGQAAKXFIXWLKX

SEQ ID NO: 25 - protein, 31 aa, Artificial Sequence
  Synthetic Peptide
XHXEGTFTSDVSSYXEGQAAKXFIXWLVKGX

SEQ ID NO: 26 - protein, 31 aa, Artificial Sequence
  Synthetic Peptide
XHXEGTFTSDVSSYXEGQAAKXFIXWLVKXX

SEQ ID NO: 27 - protein, 32 aa, Artificial Sequence
  Synthetic Peptide
XHAEGTFTSDVSSYLEGQAAKEFIAWLVKGRX

(19) United States
(12) Patent Application Publication
(10) Pub. No.: US 2006/0239933 A1
(43) Pub. Date: Oct. 26, 2006

(54) Medical product

(75) Inventors: Thomas Nilsson, Mariefred (SE); Sven Calander, Strangnas (SE); Alf Niemi, Strangnas (SE); Claes Friberg, Akers Styckebruk (SE); Lars Kax, Nykvarn (SE); Mattias Myrman, Stookholm (SE)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: MEDERIO AG, Hergiswil NW (CH)

(21) Appl. No.: 11/272,859

(22) Filed: Nov. 15, 2005

(30) Foreign Application Priority Data
Dec. 3, 2004 (SE) 0402976-5

(51) Int. Cl. A61K 9/14; A61L 9/04; A61K 38/28

(52) U.S. Cl. 424/46; 514/3; 514/12

(57) ABSTRACT

A medical product is disclosed. The medical product contains an accurately metered dose of at least one GLP medicament intended for pulmonary inhalation put into a moisture-tight, high barrier seal container. The medical product optionally also contains a dose of insulin. The container is adapted for application into a dry powder inhaler. The dose loaded in the container is intended for a prolonged delivery by inhalation to the deep lung where the active ingredients are absorbed into the system. Optionally the medical product also may comprise at least one biologically acceptable excipient.

### PRIOR APPLICATION

This application claims priority to Swedish Patent Application 0402976-5 filed Dec. 3, 2004 and U.S. patent application Ser. No. 11/049696 filed Feb. 4, 2005, both incorporated herein by reference.

### TECHNICAL FIELD

The present invention relates to a medical product comprising a metered medication dose of a glucagon-like peptide (GLP) in dry powder form and more particularly to a metered GLP dose enclosed in a sealed container adapted for use in a dry powder inhaler, capable of systemic dose delivery.

### BACKGROUND

Administering systemically acting drugs directly to the lungs of a patient by means of an inhaler is an effective, quick and user-friendly method of drug delivery, especially compared to administration by injections. A number of different inhaler devices have been developed in order to deliver drugs to the lung, e.g. pressurized aerosol inhalers (pMDIs), nebulizers and dry powder inhalers (DPIs).

The lung is an appealing site for systemic delivery of drugs as it offers a large surface area (about 100 m²) for the absorption of the molecules across a thin epithelium, thus having a potential for rapid drug absorption. Pulmonary delivery of drugs has the potential of attaining a high, rapid systemic drug concentration often without the need of penetration enhancers. The feasibility of this route of administration for a particular drug depends on, for example, dose size and extent and ease of systemic absorption through the alveols of the particular drug. The critical factors for the deposition of inhaled particles in the lung are inspiration/expiration pattern and the particle aerodynamic size distribution. The aerodynamic particle size (AD) of the drug particles is important if an acceptable deposition of the drug within the lung is to be obtained. In order for a particle to reach into the deep lung the aerodynamic particle size should typically be between 1 and 3 μm. Larger particle sizes will easily stick in the mouth and throat and will be swallowed. Thus, it is important to keep the aerodynamic particle size distribution of the dose within tight limits to ensure that a high percentage of the dose is actually deposited where it will be most effective. The aerodynamic diameter (AD) of a particle is defined as the diameter of a spherical particle having a density of 1 g/cm³ that has the same inertial properties in air as the particle of interest. If primary particles form aggregates, the aggregates will aerodynamically behave like one big particle in air.

However, finely divided powders, suitable for inhalation, are rarely free flowing but tend to stick to all surfaces they come in contact with and the small particles tend to aggregate into lumps. This is due to van der Waal forces generally being stronger than the force of gravity acting on small particles having diameters of 10 μm or less. There are several micronization technologies known in the art. Two major categories dominate in prior art: breaking of large particles using milling process such as jet milling, pearl-ball milling or high-pressure homogenization and the production of small particles using controlled production processes such as spray drying, lyophilization, precipitation from supercritical fluid and controlled crystallization. The former category produces predominantly crystalline, homogenous particles, the latter more amorphous, ‘light’, porous particles. See e.g. “Micron-Size Drug Particles: Common and Novel Micronization techniques” by Rasenack and Muller in Pharmaceutical development and technology, 2004, 9(1):1-13. See also “Unit Operation-Micronization” prepared by Lee Siang Hua, dept. of Chemical & Biomolecular Engineering, National University of Singapore. In these documents the term ‘finely divided powder’ refers to inhalable particles in general and does not limit or preclude any method of producing such particles.

Glucagon

Glucagon is a 29 amino acid peptide hormone liberated in the alpha-cells of the islets of Langerhans. It has been established that glucagon opposes the action of insulin in peripheral tissues, particularly the liver, in order to maintain the levels of blood glucose, especially if a state of hypoglycemia threatens. At mealtime, glucagon secretion is generally suppressed in healthy subjects. However, diabetics often exhibit disordered control of glucagon secretion, leading to failure to suppress hepatic glucose production and fasting hyperglycemia. Thus, it is important to determine what mechanisms are at work in relation to glucagon, so that adequate, new drugs may be produced to help the human body to function normally.

Glucagon-Like Peptide (GLP-1 and GLP-2)

GLP-1 and GLP-2 are synthesized in intestinal endocrine cells and liberated, following posttranslational processing of a single proglucagone precursor. The complex functions of these substances are not fully understood at this point and much research remains before glucagon-like peptides (GLPs) and analogues or derivates thereof can be used e.g. in the treatment of diabetes or obesity. As small and medium-sized molecules, GLPs are suitable for pulmonary delivery to the system by a dry powder inhaler, provided suitable formulations can be produced, preferably in finely divided, dry powder form.

GLP-1 exists in two principal major molecular forms, as GLP-1(7-36) amide and GLP-1(7-37). These molecules are secreted in response to nutrient ingestion and play multiple roles in metabolic homeostasis following nutrient absorption. Biological activities include stimulation of glucose-dependent insulin secretion and insulin biosynthesis, inhibition of glucagon secretion and gastric emptying and inhibition of food intake. The substance plays an important role in lowering blood glucose levels in diabetics by stimulating the beta-cells in pancreas to produce insulin. A very interesting effect of GLP-1 is that it normalizes blood glucose levels in response to hyperglycemic conditions without the risk of ending up in a hypoglycemic condition. Also, GLP-1 helps control satiety and food intake. The substance therefore constitutes an interesting pharmacological drug, particularly so for treatment of diabetes, preferably in combination with insulin or even as an alternative to a regimen of insulin. See European Patent EP 0 762 890 B1.

GLP-1 is a relatively small peptide molecule with a great potential for inhalation therapy. Fortunately, provided that the GLP-1 powder formulation is constituted of particles of the right size to sediment in the deep lung after inhalation, GLP-1 has been shown to be soluble in the fluid layer in the deep lung and dissolve, thereby ensuring rapid absorption from the lung into the system before enzymatic inactivation sets in. See for instance U.S. Pat. No. 6,720,407.

From a stability point of view, a solid formulation stored under dry conditions is normally the best choice. In the solid state, GLP molecules are normally relatively stable in the absence of moisture or elevated temperatures. GLP and analogues or derivatives thereof in dry powder form are more or less sensitive to moisture depending on the powder formulation.

GLP may be administered to humans by any available route, but oral or parenteral administration may be the most common methods in the art. Frequent injections, necessary for the management of a disease, is of course not an ideal method of drug delivery and often leads to a low patient compliance as they infringe on the freedom of the patient as well as because of psychological factors. Tablets or capsules given orally have a fairly long onset and may suffer from low efficacy because of metabolic degradation of the GLP substance before it passes into the system. Pulmonary absorption is therefore an interesting alternative, which potentially offers a fast onset, less degradation and higher efficacy. Tests have shown that users, given a choice, prefer inhalation of medicaments to self-injection.

Hence, there is a demand for precisely matched, therapeutic pulmonary dosages of GLP-based medicaments, especially in dry powder formulations and optionally in combination with insulin, and high efficacy devices for delivering dosages to the system by inhalation.

### SUMMARY OF THE INVENTION

The present invention discloses a medical product comprising an accurately metered dose of at least one GLP medicament intended for pulmonary inhalation filled in a dose container, which is effectively sealed against ingress of moisture for a specified in-use time. The medical product optionally also comprises a dose of insulin. The container is adapted for application in a dry powder inhaler. The dose loaded into the container, is intended for a prolonged delivery by inhalation to the deep lung where the active ingredients are absorbed into the system. Optionally the medical product also comprises at least one biologically acceptable excipient.

In a preferred embodiment, the present invention presents a medicament containing as active ingredient a therapeutically effective amount of a physiologically acceptable salt of at least one GLP agent including GLP analogues and derivates.

The active GLP agent exists in dry powder form suitable for administration by inhalation, optionally comprising at least one biologically acceptable excipient.

In a further aspect of the present invention the at least one GLP agent or medicament is combined with an active insulin agent, whereby the dry powder medication combination of a GLP dosage and an insulin dosage are administered by inhalation as dry powder(s) in a regimen of therapeutically effective dosages to a user in need thereof. Particularly, the combined dosages may be administered together as a single formulation, a single preparation, an inter-mixture of powders or administered separately as part-doses in a single inhalation or administered separately by separate inhalation of each part-dose.

The present invention offers the following advantages:

provides a medical product comprising an active GLP agent that is prepared in a dry powder dose for a prolonged, pulmonary delivery of the active agent by inhalation;

provides a medical product in which a well-defined dosage of an active GLP agent and optionally an insulin agent is efficiently delivered to the deep lung by a user-driven suction effort in a single inhalation process;

provides a medical product that is intended for application in a single dose inhaler, which entirely relies on the power of the inhalation for de-aggregating and aerosolizing the dose, with no further external source of power necessary; and

provides a medical product that protects the active GLP and optional insulin agents from deteriorating during a specified in-use time period.

Other advantages offered by the present invention will be appreciated upon reading of the below description of the embodiments of the invention.

### BRIEF DESCRIPTION OF THE DRAWINGS

The invention together with further objects and advantages thereof, may best be understood by making reference to the following description taken together with the accompanying drawings, in which:

FIG. 1 illustrates in a timing diagram the concentration of GLP in the system of a diabetic user after inhalation of a small dose in connection with meals during a day, compared to a big dose once a day

FIG. 2 illustrates in a timing diagram the concentration of insulin in the system of a diabetic user after inhalation of a combined dose of GLP and insulin in connection with meals during a day;

FIG. 3 illustrates in two timing diagrams a typical inhalation and dose delivery of the medical product according to the present invention;

FIG. 4 illustrates in perspective, top and side views a first embodiment of a medical product comprising a dose loaded into a high barrier seal container;

FIG. 5 illustrates in top and side views a second embodiment of a medical product comprising a dose loaded into a high barrier seal container, here illustrated in an opened state;

FIG. 6 illustrates in a top view a third embodiment of several similar medical products comprising differently sized doses loaded into identical high barrier seal containers; and

FIG. 7 illustrates in top and side views a second embodiment of a medical product comprising a combined dose loaded into two separate high barrier seal containers, adapted for insertion together into a DPI.

### DETAILED DESCRIPTION OF THE PREFERRED EMBODIMENTS

The present invention discloses an improved medical product comprising: an accurately metered medication dose of at least one active glucagon-like peptide (GLP) agent filled in a sealed container. The GLP dose is adequately protected by the sealed container from ingress of moisture for a specified in-use time period. The active GLP agent may optionally include at least one biologically acceptable excipient. The dose is intended for systemic delivery by oral inhalation and pulmonary absorption. The improved medical product is preferably adapted for a prolonged pulmonary dose delivery using a dry powder inhaler device. An objective of the present invention is to deliver an exact, high efficacy powder dosage of an active GLP agent to the system of a user via the deep lung.

The pharmacological actions of glucagon-like peptide or analogues and derivates thereof, in this document generically denoted GLP, include stimulation of insulin release, suppression of glucagon release and inhibition of gastric emptying. These actions provide one basis for this invention, where we have surprisingly found that it is possible to treat type 1 as well as type 2 diabetes by pulmonary administration of therapeutically effective amounts of GLP alone or preferably in combination with a regimen of inhalable insulin.

It will be understood by a person skilled in the art that various modifications and changes may be made to the present invention without departure from the scope thereof, which is defined by the appended claims.

In the present invention “GLP analogues” are analogues of naturally occurring GLPs (or recombinant versions), preferably human GLPs, such as GLP-1 and GLP-2, which differ by substitution of at least one naturally occurring amino acid residue with one or more other amino acid residues and/or addition/removal of at least one amino acid residue from the corresponding, otherwise identical, naturally occurring GLP. The added and/or replaced amino acid residue(s) can also be those which do not occur naturally. In this context, the number of amino acids that can be substituted, removed and/or added to the GLP sequence can non-inventively be determined by the person skilled in the art. In a preferred implementation, 1-20 amino acids of the naturally occurring GLP sequences can be replaced and/or removed, more preferably 1-10 amino acids, e.g. 1-5 amino acids. Correspondingly, in a preferred implementation, 1-20 amino acids can be added to any of the naturally occurring GLP sequences, more preferably 1-10 amino acids, e.g. 1-5 amino acids. A resulting GLP analogue is, thus, preferably a polypeptide sequence which exhibit at least about 50% sequence identity, e.g. at least 60% sequence identity, preferably at least about 70% sequence identity, more preferably at least 80%, e.g. at least 85%, 90%, 95% or 98% sequence identity the polypeptide sequence of a naturally occurring GLP. The sequence identity of two polynucleotides may be determined by several different methods known to the person skilled in the art including, but not limited to, BLAST program of Altschul et al. (J. Mol. Biol., 215: 403-410, 1990).

The important concept here is that the GLP analogue has or retains at least some of the functions of naturally occurring GLP in stimulating insulin release and biosynthesis, suppressing glucagon release and/or inhibiting gastric emptying. Any amino acid substitutions, removals or additions to the polypeptide sequence of a naturally occurring GLP that fulfils this preferred requirement of at least partly retained ∓GLP function”, as defined above, can be used to produce a GLP analogue useful according to the present invention.

“GLP derivates” are derivates of naturally occurring GLP or of a GLP analogue which are obtained by chemical modification. The chemical modification can consist, for example, in the addition, substitution or deletion of one or more specific chemical groups to one or more amino acids. It can also involve the addition, substitution or deletion of one or more chemical groups of the peptide backbone, such as, the amino and/or carboxyl terminus. Typical examples of such chemical modifications to amino acides include, without limitation, acylation of lysine ε-amino groups, N-aculation of arginine, histidine or lysine, alkylation of glutamic or aspartic carboxylic acid groups and deamidation of glutamine or asparagines. Modifications of the terminal amino include, without limitation, the des-amino, N-lower alkyl, N-di-lower alkyl and N-acyl modifications. Modification fo the terminal carboxy group include, without limitation, the amide, lower alkyl amide, dialkyl amide and lower alkyl ester modifications. Lower alkyl is C₁-C₆, and more preferably C₁-C₄ alkyl. In this context, the number of amino acids that can be modified in the GLP (analogue) sequence can non-inventively be determined by the person skilled in the art. In a preferred implementation, 1-20 amino acids can be modified, more preferably 1-10 amino acids, e.g. 1-5 amino acids.

The important concept here is that the GLP derivate has or retains at least some of the functions of naturally occurring GLP in stimulating insulin release and biosynthesis, suppressing glucagon release and/or inhibiting gastric emptying. Any amino acid modifications that fulfil this preferred requirement of at least partly retained “GLP function”, as defined above, can be used to produce a GLP derivate useful according to the present invention.

The GLP analogues and derivates useful according to the present invention can have desired new improved properties including, without limitation, improved stability, longer or shorter half-life, increased pulmonary absorption, properties that make them particular suitable for powder preparation.

Examples of suitable GLP analogues and derivates that are useful as GLP agent according to the present invention are given here below.

One particular peptide agonist acting as a GLP agent useful in the present invention is described in U.S. Pat. No. 6,528,486, which hereby is included in this document in its entirety as a reference. This GLP agent embodiment has any one of the following sequences:

| | |
|---|---|
| R₁-Gly-Glu-Gly-Thr-Phe-Thr-Ser- | (SEQ ID NOs:1-9) |
| Asp-Leu-Ser-Lys-Gln-Met-Glu-Glu- | |
| Glu-Ala-Val-Arg-Leu-Phe-Ile-Glu- | |
| Trp-Leu-Lys-Asn-Gly-Gly-Pro-Ser- | |
| Ser-Gly-Ala-R_{2,} | |

wherein

R₁— is selected from a group consisting of His- (see SEQ ID NOs: 1-3), (Lys)₆-His- (see SEQ ID NOs: 4-6) and Asn-(Glu)₅-His- (see SEQ ID NOs: 7-9) —R₂ is selected from a group consisting of -Pro-Pro-Ser-(Lys)₆ (see SEQ ID NOs: 1, 4, and 7), -Ser (see SEQ ID NOs: 2, 5, and 8) and -Ser-(Lys)₆ (see SEQ ID NOs: 3, 6, and 9).

Another particular GLP derivate, which may be used in the present invention is described in U.S. Pat. No. 6,268,343, which hereby is included in this document in its entirety as a reference. This GLP agent embodiment has any one of the following sequences:

His-Ala-Glu-Gly-Thr-Phe-Thr-Ser-Asp-Val-Ser-Ser-Tyr-Leu-Glu-Gly-Gln-Ala-Ala-R₃-Glu-Phe-Ile-Ala-Trp-Leu-Val-Arg-Gly-Arg-Gly (SEQ ID NO: 10) wherein R₃ is selected from a group consisting of Lys and Lys in which the ε-amino group is substituted with a lipophilic substituent, optionally via a spacer. Preferred lipophilic substituents include CH₃(CH₂)ₙCO—, wherein n is 6, 8, 10, 12, 14, 16, 18, 20 or 22, HOOC(CH₂)ₘCO—, wherein m is 10, 12, 14, 16, 18, 20 or 22, and lithochoyl. Preferred optional spacers include an unbranched alkane α,ω-dicarboxylic acid group having from 1 to 7 methylene groups, an amino acid residue except Cys, and γ-aminobutanoyl.

Another particular GLP derivate, a GLP-1 antagonist, which may be used in the present invention is described in US Application No. 2005/0153890, which hereby is included in this document in its entirety as a reference. This GLP agent embodiment has any one of the following sequences:

His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-R₄ (SEQ ID NOs: 11 and 12) wherein —R₄ is selected from a group consisting of -Arg (see SEQ ID NO: 11), -Arg-Gly (see SEQ ID NO: 12);

| | |
|---|---|
| His-Ser-Gln-Gly-Thr-Phe-Thr-Ser- | (SEQ ID NO:13) |
| Asp-Tyr-Ala-Lys-Tyr-Leu-Asp-Ala- | |
| Arg-Arg-Ala-Lys-Glu-Phe-Ile-Ala- | |
| Trp-Leu-Val-Lys-Cys-Arg-Gly; | |
| His-Ser-Gln-Gly-Thr-Phe-Thr-Ser- | (SEQ ID NO:14) |
| Asp-Tyr-Ala-Lys-Tyr-Leu-Asp-Ala- | |
| Arg-Arg-Ala-Lys-Glu-Phe-Ile-Ala- | |
| Trp-Leu-Val-Lys-Gly-Cys-Gly; | |
| His-Ser-Gln-Gly-Thr-Phe-Thr-Ser- | (SEQ ID NOs:15-18) |
| Asp-Tyr-Ala-R₅-Tyr-Leu-Asp-Ala- | |
| R₆-R₇-Ala-R₈-Glu-Phe-Ile-R₉-Trp- | |
| Leu-Val-R₁₀-Gly-R₁₁ | |

wherein

R₅ is selected from a group consisting of Lys, Arg, Ala

R₆ is selected from a group consisting of Arg, Lys, Ala

R₇ is selected from a group consisting of Arg, Lys

R₈ is selected from a group consisting of Lys, Ala

R₉ is selected from a group consisting of Ala, Lys

R₁₀ is selected from a group consisting of Lys, Cys, Arg

—R₁₁ is selected from a group consisting of -Arg (see SEQ ID NO: 15), -Arg-Gly (see SEQ ID NO: 16), -Arg-Cys (see SEQ ID NO: 17), -Arg-Gly-Lys (see SEQ ID NO: 18)

Other particular GLP derivates and -analogues, which may be used in the present invention are described in US2005/0014681, which hereby is included in this document in its entirety as a reference. This GLP agent embodiment is selected from a group consisting of GLP-1, GLP-1 amide, GLP-1 (7-36) amide, GLP-1 (7-37), [Val⁸]-GLP-1 (7-36) amide, [Val⁸]-GLP-1 (7-37); [Lys²⁶, ε-NH{γ-Glu(N-α-palmitoyl)}]-GLP-1 (7-37), GLP-1 (9-36) amide, GLP-1 (9-37) and GLP-2.

Another particular GLP-1 sequence, which may be used in the present invention is described in US Application No.2003/0220243, which hereby is included in this document in its entirety as a reference. This GLP agent embodiment has any one of the following sequences:

His-R₁₂-Glu-Gly-R₁₃—R₁₄-Thr-Ser-Asp-R₁₅-Ser-Ser-Tyr-Leu-Glu-R₁₆—R₁₇—R₁₈-Ala-R₁₉—R₂₀-Phe-Ile-R₂₁-Trp-Leu-R₂₂—R₂₃—R₂₄—R₂₅—R₂₆ (SEQ ID NOs: 19 and 20)

wherein

R₁₂ is selected from a group consisting of Gly, Ala, Val, Leu, Ile, Ser, Thr

R₁₃ is selected from a group consisting of Asp, Glu, Arg, Thr, Ala, Lys, His

R₁₄ is selected from a group consisting of His, Trp, Phe, Tyr

R₁₅ is selected from a group consisting of Leu, Ser, Thr, Trp, His, Phe, Asp, Val, Tyr, Glu, Ala

R₁₆ is selected from a group consisting of Gly, Asp, Glu, Gln, Asn, Lys, Arg, Cys, cysteic acid

R₁₇ is selected from a group consisting of His, Asp, Lys, Glu, Gln, Arg

R₁₈ is selected from a group consisting of Glu, Arg, Ala, Lys

R₁₉ selected from a group consisting of Trp, Tyr, Phe, Asp, Lys, Glu, His

R₂₀ is selected from a group consisting of Ala, Glu, His, Phe, Tyr, Trp, Arg, Lys

R₂₁ is selected from a group consisting of Ala, Glu, Asp, Ser, His

R₂₂ is selected from a group consisting of Asp, Arg, Val, Lys, Ala, Gly, Glu

R₂₃ is selected from a group consisting of Glu, Lys, Asp

R₂₄ is selected from a group consisting of Thr, Ser, Lys, Arg, Trp, Tyr, Phe, Asp, Gly, Pro, His, Glu

R₂₅ is selected from a group consisting of Thr, Ser, Asp, Trp, Tyr, Phe, Arg, Glu, His

—R₂₆ is selected from a group consisting of -Lys, -Arg, -Thr, -Ser, -Glu, -Asp, -Trp, -Tyr, -Phe, -His, -NH₂, -Gly, -Gly-Pro (see SEQ ID NO: 20), -Gly-Pro-NH₂ (see SEQ ID NO: 20) or is deleted.

A particular peptide agonist acting as a GLP agent useful in the present invention is described in U.S. Application No. 2003/0199672. This GLP agent embodiment has any one of the following sequences:

| | |
|---|---|
| His-R₂₇-R₂₈-Gly-R₂₉-Phe-Thr-R₃₀-Asp- | (SEQ ID NO:21) |
| R₃₁-R₃₂-R₃₃-R₃₄-R₃₅-R₃₆-R₃₇-R₃₈-R₃₉- | |
| R₄₀-R₄₁-R₄₂-Phe-Ile-R₄₃-R₄₄-R₄₅-R₄₆- | |
| R₄₇-R₄₈-R₄₉-R₅₀-R₅₁-R₅₂-R₅₃-R₅₄-R₅₅- | |
| R₅₆-R₅₇-R₅₈ | |

wherein

R₂₇ is selected from a group consisting of Ala, Gly, Ser, Thr, Leu, Ile, Val, Glu, Asp, Lys

R₂₈ is selected from a group consisting of Glu, Asp, Lys

R₂₉ is selected from a group consisting of Thr, Ala, Gly, Ser, Leu, Ile, Val, Glu, Asp, Lys

R₃₀ is selected from a group consisting of Ser, Ala, Gly, Thr, Leu, Ile, Val, Glu, Asp, Lys

R₃₁ is selected from a group consisting of Val, Ala, Gly, Ser, Thr, Leu, Ile, Tyr, Glu, Asp, Lys

R₃₂ is selected from a group consisting of Ser, Ala, Gly, Thr, Leu, Ile, Val, Glu, Asp, Lys

R₃₃ is selected from a group consisting of Ser, Ala, Gly, Thr, Leu, Ile, Val, Glu, Asp, Lys

R₃₄ is selected from a group consisting of Tyr, Phe, Trp, Glu, Asp, Lys

R₃₅ is selected from a group consisting of Leu, Ala, Gly, Ser, Thr, Leu, Ile, Val, Glu, Asp, Lys

R₃₆ is selected from a group consisting of Glu, Asp, Lys

R₃₇ is selected from a group consisting of Gly, Ala, Ser, Thr, Leu, Ile, Val, Glu, Asp, Lys

R₃₈ is selected from a group consisting of Gln, Asn, Arg, Glu, Asp, Lys

R₃₉ is selected from a group consisting of Ala, Gly, Ser, Thr, Leu, Ile, Val, Arg, Gln, Asp, Lys

R₄₀ is selected from a group consisting of Ala, Gly, Ser, Thr, Leu, Ile, Val, Glu, Asp, Lys

R₄₁ is selected from a group consisting of Lys, Arg, Gln, Asp, His

R₄₂ is selected from a group consisting of Gln, Asp, Lys

R₄₃ is selected from a group consisting of Ala, Gly, Ser, Thr, Leu, Ile, Val, Glu, Asp, Lys

R₄₄ is selected from a group consisting of Trp, Phe, Tyr, Glu, Asp, Lys

R₄₅ is selected from a group consisting of Leu, Gly, Ala, Ser, Thr, Ile, Val, Glu, Asp, Lys

R₄₆ is selected from a group consisting of Val, Gly, Ala, Ser, Thr, Leu, Ile, Glu, Asp, Lys

R₄₇ is selected from a group consisting of Lys, Arg, Glu, Asp, His

R₄₈ is selected from a group consisting of Gly, Ala, Ser, Thr, Leu, Ile, Val, Glu, Asp, Lys

R₄₉ is selected from a group consisting of Arg, Lys, Glu, Asp, His

R₅₀ is selected from a group consisting of Gly, Ala, Ser, Thr, Leu, Ile, Val, Glu, Asp, Lys or is deleted

R₅₁ is selected from a group consisting of Arg, Lys, Glu, Asp, His or is deleted

R₅₂ is selected from a group consisting of Arg, Lys, Glu, Asp, His or is deleted

R₅₃ is selected from a group consisting of Asp, Glu, Lys or is deleted

R₅₄ is selected from a group consisting of Phe, Trp, Tyr, Glu, Asp, Lys or is deleted

R₅₅ is selected from a group consisting of Pro, Lys, Glu, Asp or is deleted

R₅₆ is selected from a group consisting of Glu, Asp, Lys or is deleted

R₅₇ is selected from a group consisting of Glu, Asp, Lys or is deleted

—R₅₈ is selected from a group consisting of-Val, -Glu, -Asp, -Lys or is deleted

Another particular GLP-1 sequence, which may be used in the present invention is described in PCT Application No. WO2005/066207. This GLP agent embodiment has any one of the following sequences:

R₅₉-His-R₆₀-Glu-Gly-Thr-Phe-Thr-Ser-Asp-Val-Ser-Ser-Tyr-R₆₁-Glu-Gly-Gln-Ala-Ala-Lys-R₆₂-Phe-Ile-R₆₃-Trp-Leu-R₆₄ (SEQ ID NOs: 22-26)

wherein

R₅₉ is selected from a group consisting of H, a linear or branched unsaturated C₁-C₆ acyl group, an optionally substituted arylcarbonyl, an optionally cycloalkylcarbonyl, an optionally substituted arylalkylcarbonyl

R₆₀ is selected from a group consisting of Ala, 1-aminoisobutyric acid (Aib), Val, Gly

R₆₁ selected from a group consisting of Leu and Gly having a C₆-C₂₀ alkyl side chain

R₆₂ is selected from a group consisting of Ala, Leu, Val, Ile, Glu

R₆₃ is selected from a group consisting of Glu, Asp, Asn, Gln, Ala

—R₆₄ is selected from a group consisting of -Lys-Asn-Aib-OH (see SEQ ID NO: 22), -Lys-Asn-Aib-NH₂ (see SEQ ID NO: 22), -Val-Lys-Asn-OH (see SEQ ID NO: 23), -Val-Lys-Asn-NH₂ (see SEQ ID NO: 23), -Lys-Asn-OH (see SEQ ID NO: 24), -Lys-Asn-NH₂ (see SEQ ID NO: 24), -Val-Lys-Gly-Arg-NH₂ (see SEQ ID NO: 25), -Val-Lys-Aib-Arg-OH (see SEQ ID NO: 26), -Val-Lys-Aib-Arg-NH₂ (see SEQ ID NO: 26), -Lys-Asn-Gly-OH (see SEQ ID NO: 22), -Lys-Asn-Gly-NH₂ (see SEQ ID NO: 22)

Another particular GLP-1 sequence, which may be used in the present invention is described in PCT Application No. WO2004/029081. This GLP agent embodiment has any one of the following sequences:

| | |
|---|---|
| R₆₅-His-Ala-Glu-Gly-Thr-Phe-Thr-Ser- | (SEQ ID NO:27) |
| Asp-Val-Ser-Ser-Tyr-Leu-Glu-Gly-Gln- | |
| Ala-Ala-Lys-Glu-Phe-Ile-Ala-Trp-Leu- | |
| Val-Lys-Gly-Arg-R₆₆ | |

wherein

R₆₅ is a rigidifying hydrophobic moiety selected from the group consisting of

C₁-C₁₀ alkenoic acid, optionally substituted by at least one substituent selected from the group consisting of straight or branched C₁-C₆ alkyl, C₃-C₆ cycloalkyl, aryl and substituted aryl;

C₁-C₁₀ alkynoic acid;

C₃-C₁₀ cycloalkanoic acid, or heterocycloalkanoic acid comprising an heteroatom selected from O, S and N;

C₅-C₁₄ arylcarboxylic or arylalkanoic acid optionally substituted by at least one substituent selected from the group consisting of lower alkyl, lower alkoxy, lower alkylthio, halo, hydroxy, trifluoromethyl, amino, —NH(lower alkyl), —N(lower alkyl)₂, di- and tri-substituted phenyl, 1-naphtyl and 2-naphtyl substituted with a substituent selected from the group consisting of methyl, methoxy, methylthio, halo, hydroxy and amino;

C₅-C₁₄ heteroarylcarboxylic or heteroarylalkanoic acid comprising a heteoatom selected from O, S and N, and being optionally substituted by at least one substituent selected from the group consisting of lower alkyl, lower alkoxy, lower alkylthio, halo, hydroxy, trifluoromethyl, amino, —NH(lower alkyl), —N(lower alkyl)₂, di- and tri-substituted phenyl, 1-naphtyl and 2-naphtyl substituted with a substituent selected from the group consisting of methyl, methoxy, methylthio, halo, hydroxy and amino

—R₆₆ is selected from a group consisting of —OH, —NH₂, -Gly-OH.

In a particular aspect of the present invention a GLP agent is selected, which is long-acting following pulmonary delivery. In all embodiments herein, more than one GLP agent can be used.

In a particular aspect of the present invention a GLP medicament is used as an alternative to subcutaneous insulin in the treatment of early diabetes type 2, where a regimen of the GLP medicament, optionally in combination with insulin, through a pulmonary route of administration eliminates the use of subcutaneous insulin to a user.

In a further aspect of the present invention a GLP medicament is used in combination with insulin in the treatment of diabetes type 1 and 2, such that a regimen of inhaled GLP and insulin for instance in connection with meals three or four times per day is well adapted to the needs of a diabetic user with the objective of improving glycemic control for the user and eliminating subcutaneous insulin altogether.

Self-administration of peptides, such as insulin, by subcutaneous injection is part of everyday life for many patients with diabetes. Normally, the user needs to administer insulin several times daily based on close monitoring of the glucose level. Incorrect timing of the administration or incorrect dosing may lead to hyperglycemia or hypoglycemia. Also, there are pharmacokinetic limitations when using the subcutaneous route. Absorption of insulin after a subcutaneous injection is slow. It sometimes takes up to an hour before the glucose level in the blood begins to be significantly reduced. This inherent problem with subcutaneous insulin delivery cannot be solved with a more frequent administration. In order to obtain plasma insulin concentrations that are physiologically correct over time it is advantageous to choose another route of administration, such as inhalation.

In yet another particular aspect of the present invention GLP, administered by inhalation for pulmonary absorption into the system, optionally in combination with insulin, improves user quality of life and user compliance with a prescribed dosing regimen based on inhalation of medicaments, compared to injections or a mixture of oral administration and injections. Systemic delivery by pulmonary absorption is faster and more accurate than by subcutaneous injection, partly because of the difficulty in the latter method to control exactly where the dose will be located in the subcutaneous tissue and as a consequence the systemic concentration over time will vary considerably from one injection to the next. Furthermore, GLP has a rather small therapeutic window, i.e. a too small dose will have no effect at all whereas a too big dose will often cause the user to feel sick and even cause the user to vomit. The pulmonary route for GLP is thus to be preferred because of fast on-set, exactness, user comfort and reduced adverse side effects.

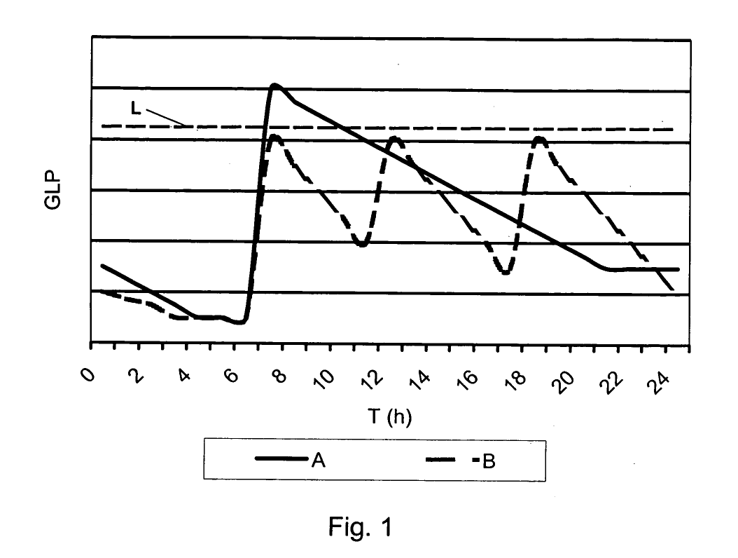

Advantageously, GLP is inhaled several times daily in connection with meals, so that the GLP effect on the pancreatic insulin production is not too small nor leading to too high concentration in the blood, but so that the GLP concentration is kept within the optimal therapeutic window, thereby leading to a better control of glucose concentration in the blood. See FIG. 1, showing two curves, A and B over time T, representing plasma concentration of GLP, where curve A is the result of a single, high dose administered in the morning compared to 3 smaller doses administered in direct connection with meals during the day as in curve B. Curve. A shoots over the permitted maximum level L, which causes unwanted adverse effects in a subject, such as nausea or inducing vomiting attacks. Clearly, a better way to achieving glycemic control is to administer GLP in relatively small doses in connection with meals.

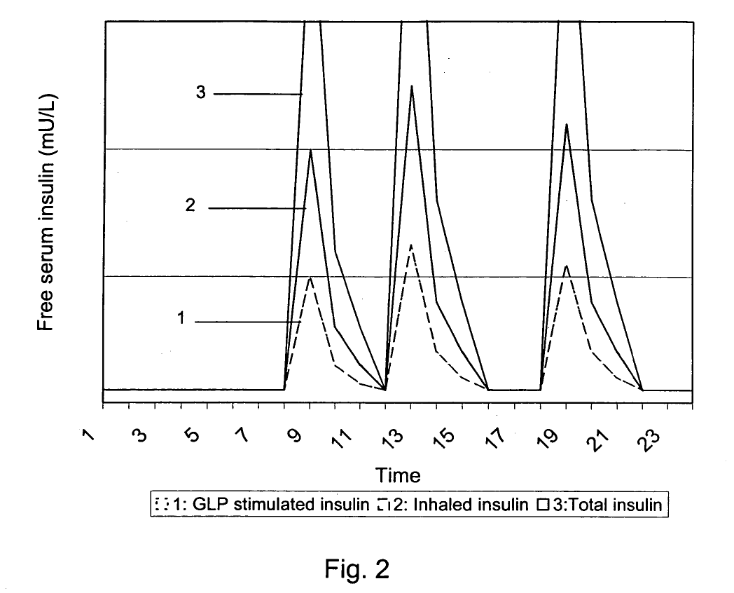

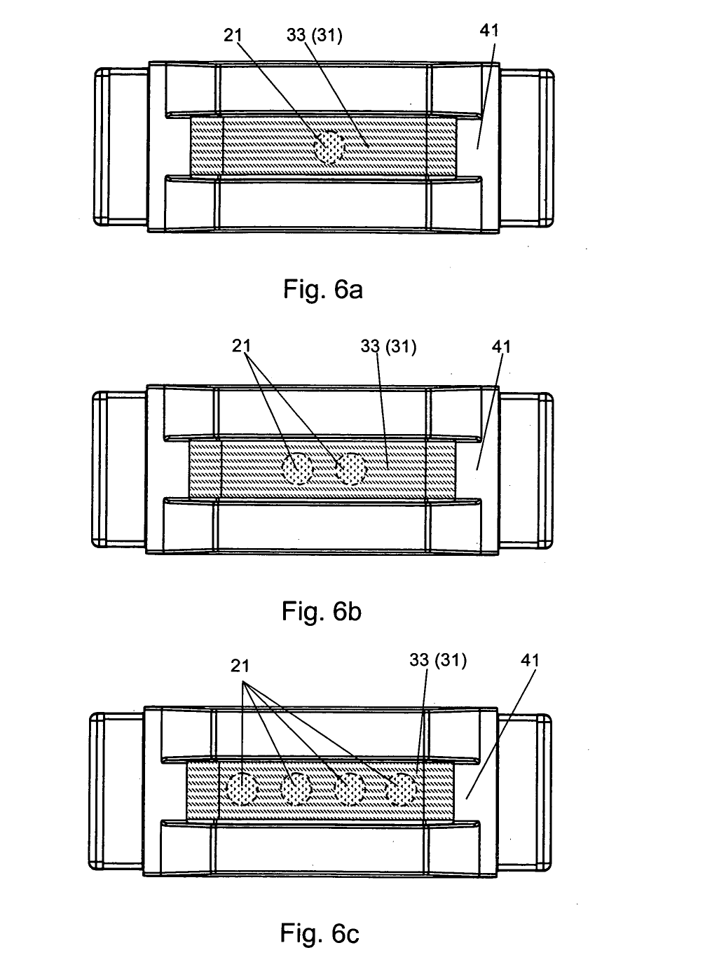

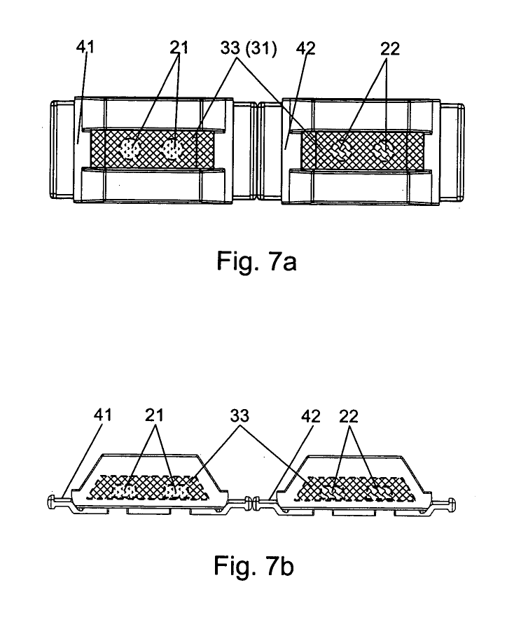

In a particular embodiment of the present invention the medical product is arranged such that a selected, effective dose of GLP is combined with a dose of insulin, where the size of the insulin dose is selected before each administration by a diabetic user based on an estimation or actual measurement of the present level of glucose in the blood and with a regard for the imminent meal. A dry powder inhaler is thus to be loaded by the said user with a sealed container carrying a dose of GLP and the same or a similar container carrying a titratable dose of insulin, e.g. containing the equivalence of from 1 to 100 insulin units (IU). Thus, a therapeutically effective insulin dose mass is normally in a range from 100 μg to 25 mg. Both doses are then administered in a single inhalation. See FIGS. 7a and 7b illustrating two carriers, 41 and 42, each carrying a sealed container 33 (seal 31) containing a dose 21 of GLP and a dose 22 of insulin respectively. The doses are hidden from view by the respective sealed container, but nevertheless indicated in the illustration for the benefit of the reader. For instance, the user has been supplied with a number of identical GLP dose containers and a collection of insulin dose containers representing three different dose sizes, low, medium and high, plus empty dose containers. For example, differently sized doses 21 may be loaded into identical or similar sealed containers 33 (seal 31) and fitted to carriers 41 as illustrated in FIGS. 6a, 6b and 6c. Based on the need of the user in the course of a day, he or she decides, e.g. based on a measurement of blood sugar level, what combination is required at each instance of administration and composes an adequate combination of GLP and insulin, where the GLP dose is fixed but the insulin dose is variable. The flexibility of the medical product will permit GLP to stimulate the self production of insulin and only add a minimum of exogenous insulin to help control blood sugar. See FIG. 2 for graphic representations of insulin plasma concentration partly from GLP stimulated endogenous insulin 1, exogenous insulin 2 and the combined insulin concentration 3 over time during a day, if a combined dose of GLP and insulin is administered in connection with meals.

In another embodiment of the invention a GLP dose is loaded in the same dose container as a dose of insulin, and the combined doses are then delivered by a dry powder inhaler in a single inhalation from the single dose container. This embodiment is possible providing the GLP and the insulin do not detrimentally affect each other during transport and storage. See our U.S. Application No. 2004/0258625, which is hereby included by reference.

There are many advantages in combining GLP and insulin in a medical product intended for administration by inhalation in the treatment of diabetes 1 and 2, such as:

Substantial reduction of insulin doses is possible

Big improvement in glycemic control

Endogenous insulin secretion is stimulated

Risk of hyperglycemia is substantially reduced

Partial or complete inhibition of insulin injections is possible

Less adverse side effects

Big improvement in user quality of life

Better user compliance

In short, a combined therapy comprising GLP and insulin results in better medical status and higher quality of life for the user.

Besides diabetes 1 and 2, other important and interesting therapeutic areas, where GLP may be a highly effective drug, especially in combination with other medicaments, such as insulin, are cardiovascular disorders, conditions of obesity, dyslipidemia and lipodystrophy.

From the disclosure herein, however, it is clear that the quality of a delivered GLP dose, as well as an insulin dose, to the lung needs to be very high in terms of fine particle fraction. As has been pointed out in the foregoing, particles need to be 5 μm or less in aerodynamic diameter (AD) to have a reasonable chance of reaching into the deep lung when inhaled. Large particles may impact and stick in the mouth or further down in the airways before they reach the deep lung. In the deep lung, small particles may be absorbed by the alveoli and delivered to the system. AD of particles should preferably be in a range from 0.5 to 5 μm and more preferably in a range 1 to 3 μm for a rapid and successful delivery to the system through the lung. Particles of this size sediment in the lung provided that the inhalation is deep and not too short. For maximum lung deposition, the inspiration must take place in a calm manner to decrease air speed and thereby reduce deposition by impaction in the upper respiratory tracts. Small particles are more easily absorbed by the alveoli, which is a further reason for the delivered dose, according to the disclosure, to present a high fine particle fraction (FPF), i.e. the fine particle dose (FPD) of the delivered dose mass should be as high as possible.

The advantages of using the inhalation power of the user to full potential in a prolonged, continuous dose delivery interval within the inhalation cycle is disclosed in our U.S. Pat. No. 6,622,723 (WO 01/34233 A1), which is hereby incorporated herein by reference in its entirety. An objective of a prolonged dose delivery is to achieve a very high level of particle de-aggregation when the dose is in the process of being released from the container where it is deposited. In a preferred embodiment of the present invention, the medical product is optimized for a prolonged dose delivery.

Prior art dry powder inhalers begin aerosolizing a dose by uncontrolled spreading of energy to the powder in the dose. In prior art the supplied energy may be of different kinds, e.g. mechanical, electric or pneumatic to name a few and combinations of different kinds are common, e.g. where the inhalation energy provided by the user is re-enforced by external sources of power to accomplish particle de-aggregation and aerosolization of the dose. But the energy thus provided is directed to the whole dose for a short time. Surprisingly, we have found that the energy thus provided becomes unevenly distributed onto and in the dose, i.e. the energy density (Ws/m³) is too low in parts of the dose for de-aggregation to come about. Thus, significant parts of the dose are aerosolized as aggregated particles and delivered as aggregates to a user. However, these aggregates are aerodynamically too big to reach the deep lung. This is why the delivered fine particle doses (FPD) out of blisters or capsules or aerosolizing chambers of prior art inhalers are too low, representing only 20-30% of the metered dose mass.

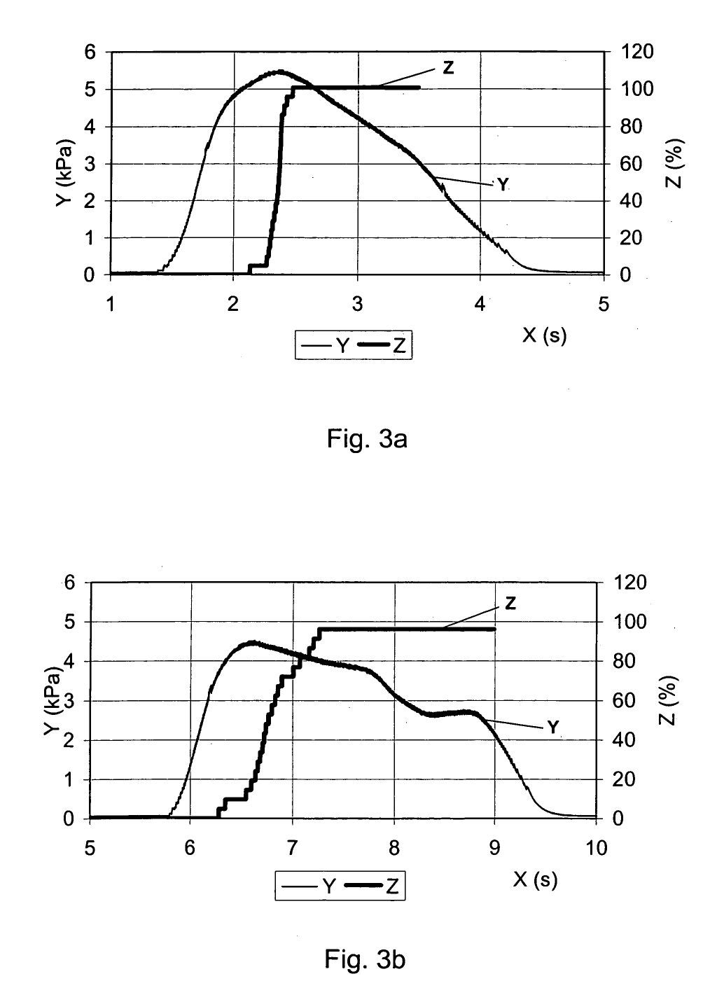

According to the present invention, a particular solution to this problem of individually releasing all particles of the dose, is to optimize the use of available inhalation energy over time. An initial build-up of suction power establishes an airflow, which is then directed onto the dose in a piecemeal fashion. The particles in the dose are thus released and aerosolized by the high level of energy density (Ws/m³) supplied to the dose in a gradual manner. Thus, a preferred embodiment of the medical product is adjusted to accommodate and facilitate a gradual release of the enclosed GLP dose and an optional dose of insulin by a dry powder inhaler. Surprisingly, we have found that if the inhalation power of a user is first allowed to build up to a certain level and then applied for a prolonged period to a single or combined dose, no other external source of power is necessary for a complete release and aerosolization of the dose(s). A minimum level of power has been determined to be 2 kPa of suction and a normal range of suction power is 2 to 6 kPa, but typically a suction not less than 2 kPa and not greater than 4 kPa is quite satisfactory for complete particle by particle release of a single or combined dose. Preferably, the suction produces an inspiration air stream in a range 20 to 60 l/min and more preferably in a range 20 to 40 l/min. Arranging the medical product, according to the invention, for a prolonged delivery in this way results in an FPD value several times higher than in prior art. Since the dose is aerosolized gradually, the dose is delivered over an interval, thereby resulting in a prolonged pulmonary dose delivery. Typically, a prolonged pulmonary dose delivery lasts from 0.1 s to 5 s, depending on dose mass in the medical product and design and efficiency of the dry powder inhaler that is used. Two typical inhalation sequences are illustrated in FIGS. 3a and 3b, carried out by two subjects. Diagram curve Y represents the suction power in kPa provided by the respective subject over time X and curve Z represents dose delivery from 0 to 100% from a DPI. As can be seen, delivery of the dose does not begin until the suction is near the peak at about 4 to 5 kPa. The respective dose is fully delivered before the suction power has dropped below 4 kPa. In one embodiment of the invention the medicament dose is made available in a dry powder inhaler and a user provides the suction effort to the inhaler, whereby the dose is released in a resulting single inhalation operation. In another embodiment of the invention the medicament dose is made available in a dry powder inhaler and a machine operated means provides the suction effort to the inhalation operation whereby the dose is released and pulmonary delivery is mimicked by a mechanical in-vitro means.

In a preferred embodiment of the present invention the prolonged delivery is accomplished within a time period of not less than 0.1 second and not more than 5 seconds by the inhaler device.

In another embodiment of the present invention the prolonged delivery is accomplished within a time period of not less than 0.2 second and not more than 2 seconds by the inhaler device.

In a different embodiment of the present invention the prolonged delivery is accomplished within a time period of not less than 0.2 seconds and not more than 5 seconds and the dose is delivered in a manner where at least 50% of the dose by mass is emitted within a time frame of 0.2-1 seconds by the inhaler device.

In yet another embodiment of the present invention the prolonged delivery is accomplished within a time period of not less than 0.2 seconds and not more than 5 seconds and the dose is delivered in a manner where at least 75% of the dose by mass is emitted within a time frame of 0.2-2 seconds by the inhaler device.

Surprisingly, we have found that aerosolizing the dose gradually leads to less irritation of the mucous membranes and airways of the user, with a reduced risk of coughing or choking during an inhalation. This beneficial effect is due to a reduced concentration of particles per liter inspiration air, compared to prior art combinations of dose packages and inhalers. In a further aspect of the present invention the medical product is intended for application in a single dose inhaler, which entirely relies on the power of the inhalation for de-aggregating and aerosolizing the dose, with no further external source of power necessary. See FIGS. 7a and 7b for an example of a medical product comprising a combination of GLP and selectable insulin doses.

The disclosure herein is by way of example and a person of ordinary skill in the art may of course find alternative methods of energy optimization, whereby de-aggregation power of sufficient strength may be distributed evenly and efficiently onto the dose, which methods, however, are still within the scope of the present invention. See our U.S. Pat. Nos. 6,571,793, 6,881,398, 6,840,239 and 6,892,727, which are hereby incorporated herein by reference.

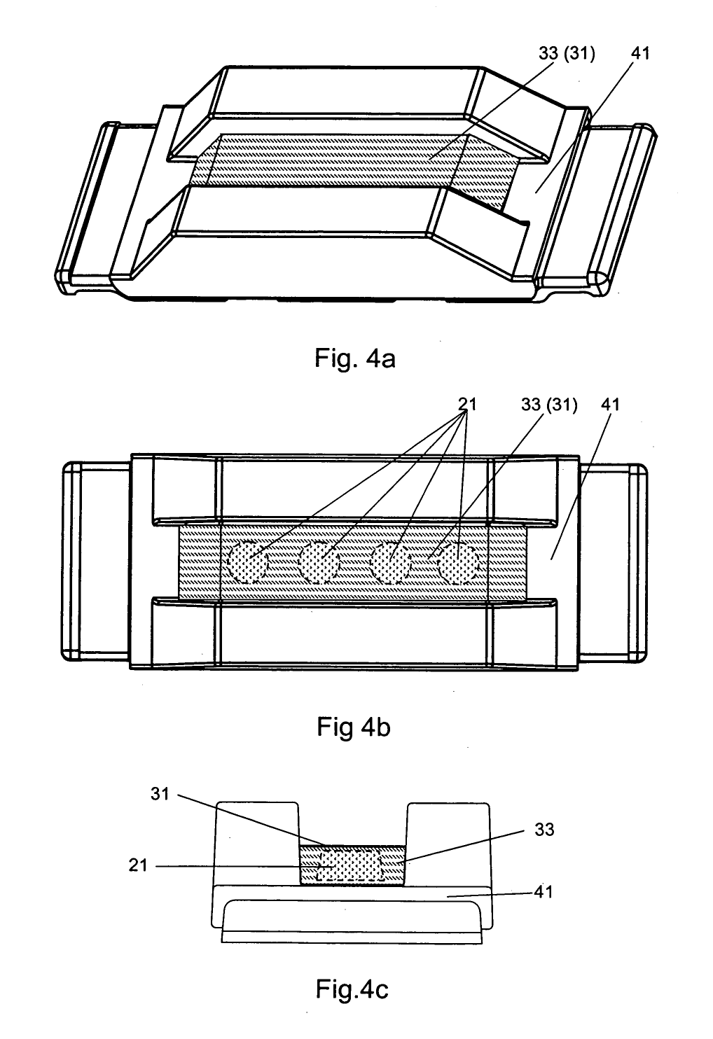

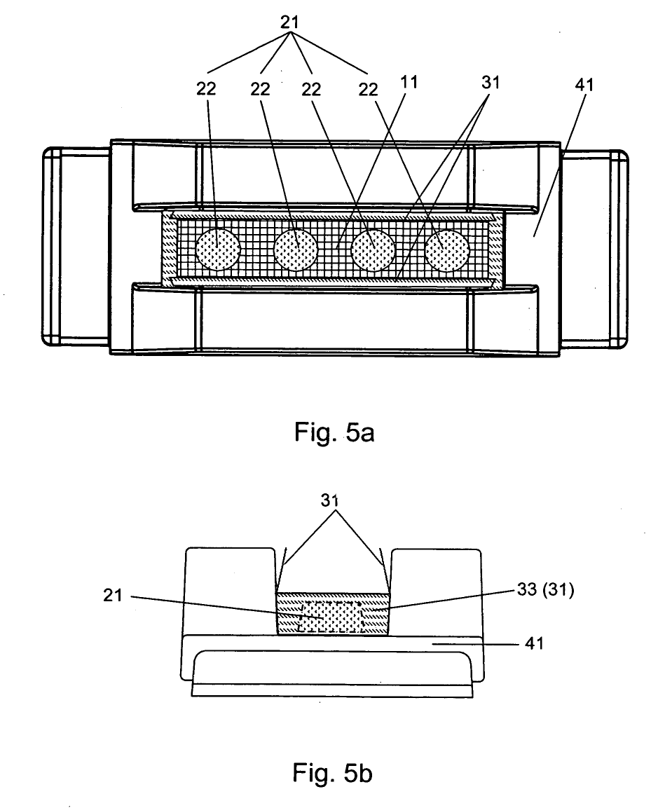

In another aspect of the invention it is important to protect a moisture-sensitive dose, such as GLP or insulin, up to the very point of delivery to a user. Therefore, the medical product of the present invention must be protected from ingress of moisture for a specified in-use period. Preferably, the container of the medical product of the present invention is not opened until a user performs an inhalation. In such case the time of exposing the dose powder to the atmosphere is approximately the time it takes for the delivery to take place. Any adverse effect, which depends on exposing the dose to the ambient atmosphere is thereby minimized and in practice negligible. A particular embodiment of the present invention is illustrated in FIGS. 4a, 4b and 4c. FIG. 4a shows a sealed container 33 (seal 31) put into a protective carrier 41 adapted for insertion into a dry powder inhaler. FIG. 4b shows a top view of the carrier/container and indicates depositions of dry powder making up a metered dose inside the container 33 under a seal 31, for the benefit of the reader. FIG. 4c illustrates a side view of the carrier/container in FIG. 4b. FIGS. 5a and 5b illustrate the container 33 in an opened state, where the seal 31 has been slit open and folded upwards, away from the dose 21 inside the container 33. Dose 21 is in the embodiment made up of four separate depositions 22 of dry powder. Depositions 22 may comprise same or different powders, such that the combined depositions either represent a single, metered GLP dose or a combined dose of GLP and insulin. A skilled person would realize that the number of depositions depends, inter alia, on the total dose mass and the relation between masses of different powders together making up a combined dose.

The fine particle fraction (FPF) of the finely divided active peptide agent, GLP and optionally insulin, if present, in the metered medicament dose is to be as high as possible, having a mass median aerodynamic diameter (MMAD) below 3 μm and a particle size distribution having at least 70% and preferably more than 80% and most preferably more than 90% by mass with AD between 1 and 3 μm. After forming a metered dose, it is very important to protect the dose from negative influences, which may otherwise detrimentally affect FPF of GLP as well as insulin. Moisture constitutes a particular risk in this respect, because moisture increases the tendency of powders to form agglomerates, which reduces the FPF of the powder. So, in order to protect the dose according to the present invention against moisture, the medical product either comprises a primary dose package constituting a high barrier seal container, or the medical product is put in a suitable secondary package, whereby the FPF of GLP as well as optional insulin is protected from ingress of moisture from the point of manufacture to the point of administering a dose, through the steps of transporting, storing, distributing and consuming.

Methods of dose forming of peptide powder formulations, e.g. GLP and insulin according to the present invention, include conventional mass, gravimetric or volumetric metering and devices and machine equipment well known to the pharmaceutical industry for filling blister packs, for example. Electrostatic forming methods may also be used, or combinations of methods mentioned. A most suitable method of depositing microgram and milligram quantities of dry powders uses electric field technology (ELFID) as disclosed in our U.S. Pat. No. 6,592,930 B2, which is hereby incorporated in this document in its entirety as a reference.

Insulin according to the present invention is defined as insulin, insulin analogue and insulin derivates, preferably recombinant, human insulin. “Insulin analogues” are analogues of naturally occurring insulin, namely human insulin or recombinant human insulin, which differ by substitution of at least one naturally occurring amino acid residue with other amino acid residue(s) and/or addition/removal of at least one amino acid residue from the corresponding, otherwise identical, naturally occurring insulin. The added and/or replaced amino acid residue(s) can also be those which do not occur naturally. In this context, the number of amino acids that can be substituted, removed and/or added to the insulin sequence can non-inventively be determined by the person skilled in the art. In a preferred implementation, 1-30 amino acids of the naturally occurring insulin sequences can be replaced and/or removed, preferably 1-20 amino acids, and more preferably 1-10 amino acides, e.g. 1-5 amino acids. Correspondingly, in a preferred implementation, 1-30 amino acids can be added to the naturally occurring insulin sequence, preferably 1-20 amino acids, and more preferably 1-10 amino acids, e.g. 1-5 amino acids. A resulting insulin analogue is, thus, preferably a polypeptide sequence which exhibit at least about 50% sequence identity, e.g. at least 60% sequence identity, preferably at least about 70% sequence identity, more preferably at least 80%, e.g. at least 85%, 90%, 95% or 98% sequence identity the polypeptide sequence of a naturally occurring insulin.

The important concept here is that the insulin analogue has or retains at least some of the functions of naturally occurring insulin in stimulating uptake, storage and use of glucose by almost all tissues of the body, but especially by the muscles, adipose tissue and liver. Any amino acid substitutions, removals or additions to the polypeptide sequence of naturally occurring insulin that fulfils this preferred requirement of at least partly retained “insulin function”, as defined above, can be used to produce an insulin analogue useful according to the present invention.

“Insulin derivates” are derivates of naturally occurring insulin or of an insulin analog which are obtained by chemical modification. The chemical modification can consist, for example, in the addition, substitution or deletion of one or more specific chemical groups to one or more amino acids. It can also involve the addition, substitution or deletion of one or more chemical groups of the peptide backbone, such as, the amino and/or carboxyl terminus. Typical examples of such chemical modifications include, without limitation, acylation of lysine ε-amino groups, N-aculation of arginine, histidine or lysine, alkylation of glutamic or aspartic carboxylic acid groups and deamidation of glutamine or asparagines. Modificaiton of the terminal amino include, without limitation, the des-amino, N-lower alkyl, N-di-lower alkyl and N-acyl modifications. Modification of the terminal carboxy group includes, without limitation, the amide, lower alkyl amide, dialkyl amide and lower alkyl ester modifications. Lower alkyl is C₁-C₆, and more preferably C₁-C₄ alkyl.

In this context, the number of amino acids that can be modified in the insulin (analogue) sequence can non-inventively be determined by the person skilled in the art. In a preferred implementation, 1-30 amino acids can be modified, more preferably 1-20 or 1-10 amino acids, e.g. 1-5 amino acids.

The important concept here is that the insulin derivate has or retains at least some of the functions of naturally occurring insulin in stimulating uptake, storage and use of glucose by almost all tissues of the body. Any amino acid modifications that fulfills this preferred requirement of at least partly retained “insulin function”, as defined above, can be used to produce an insulin derivate useful according to the present invention.

The insulin analogues and derivates useful according to the present invention can have desired new improved properties including, without limitation, improved stability, longer or shorter half-life, increased pulmonary absorption, properties that make them particular suitable for powder preparation.

Prior art methods of producing a powder formulation of a medicament intended for inhalation, such as insulin or GLP, generally involves micronizing e.g. by jet milling or spray-drying, freeze-drying, vacuum drying or open drying. Prior art methods include the addition of excipients, e.g. surfactants, stabilizers and penetration enhancers, in the manufacturing process with the object of improving the bioavailability, speed of systemic absorption and efficacy of the medicament, for instance insulin. Methods also include making porous or hollow particles, preferably spherical in shape and geometrically bigger than 10 μm in diameter, but with AD less than 5 μm. The objectives are to get a flowable powder, which makes handling and dose forming and metering easier and yet to provide a powder, which is easy to de-aggregate when inhaled and which offers a high delivered FPD.

A particular method of preparing a dry, crystalline medicament powder before an optional mixing step, is to jet mill or otherwise micronize the ingredients of the medicament at least once and preferably twice in order to get a small mass median aerodynamic diameter (MMAD) for the finely divided powder in a range 1-3 μm with as small tails of particles outside this range as possible. The powder is then optionally mixed with one or more excipients, for example in order to dilute the potency of the active ingredient(s) to get a final powder preparation well adapted to chosen methods of metering and forming doses.

In another aspect of the present invention of combining GLP and insulin in treatment of diabetes, it is advantageous to include more than one formulation of recombinant, human insulin, or human insulin analogue, powder in the insulin dose, e.g. in order to improve the insulin delivery into the blood circulation, such that the natural course of insulin production in a healthy person is mimicked more closely than would be possible when using only one insulin formulation. Different formulations of recombinant insulin and insulin analogue present different absorption delays and blood concentrations over time, e.g. Lantus from Sanofy-Aventis, which is slow-acting but long duration and insulin lispro Humalog from Eli Lilly, the latter having fast on-set. Therefore, a use of two or more insulin analogues in a combined dose with GLP is well suited with the objective of adjusting the systemic concentration of insulin in the blood of a diabetic user over time by the combined action of the active ingredients. This treatment comes very close to bringing about the natural concentration curve in a healthy subject. When insulin is combined with administration of GLP, the choice of suitable insulin formulations and dosage sizes must be carefully adjusted by a person skilled in the art for best possible combination result. A typical combined therapy and dosing regimen of GLP and insulin lets the diabetic user take a combined dose by inhalation just before or in connection with each meal, such as breakfast, lunch and dinner. The insulin and the GLP ingredients are within minutes of inhalation absorbed into the system. The insulin helps reduce the spike of glucose following intake of food and the GLP stimulates the beta-cells in pancreas to produce insulin and helps the body to keep a normal level of glucose in the blood until it is time for the next meal. In this therapy the objective of controlling a normal glucose level in the user during the day is fulfilled. Optionally, depending on the diabetic status of the user, additional doses of GLP and/or insulin may be required in order to control the level of glucose during the day and night.

According to the present invention, mixing of two or more active agents into a homogenous powder mixture, optionally including one or more excipients, may be done in any order of all possible permutations, before the resulting powder mixture is used in a method of metering and forming doses. For instance, insulin may be mixed with GLP first and then this mixture may be added to a mixture of excipients, if needed, but any permutation of the mixing steps may be used. The properties of the final powder mixture are decisive for the choice of mixing method, such that e.g. peptide stability is maintained, risk of particle segregation by size is eliminated and dose to dose relative standard deviation (RSD) is kept within specified limits, usually within 5%. Naturally, the ingredients must not adversely affect each other in the mixture. If there is any risk of degradation or other adverse effect in a component resulting from the mixing, then that component must not be included in the mixture, but separately administered, although preferably in a single inhalation operation, if technically possible.

In another aspect of the present invention separate dry powder dosages of GLP and insulin respectively, each optionally comprising excipients, may be arranged onto a common dose carrier for insertion into an adapted inhaler and delivered to the lungs of a user, preferably in the course of a single inhalation. In a particular embodiment the separated dosages are separately enclosed onto the dose carrier in individually sealed enclosures, such as compartments, containers, capsules or blisters, known in the art. In another embodiment the separated dosages share a common enclosure onto the dose carrier. A common, sealed enclosure may be used to simplify the manufacturing process if the dosages of GLP and insulin have no adverse effect on each other after deposition and sealing onto the carrier for the shelf-life of the product. The combined dosages according to the disclosure may be advantageously used in the treatment of diabetes type 1 and type 2, providing at least one of the advantages listed in the foregoing.

It is a further objective of the present invention to deliver a fine particle dose (FPD) of the at least one GLP powder and optionally insulin powder if included in a combined dose, where the delivered fine particle dose amounts to at least 50% by mass, preferably at least 60% by mass, more preferably at least 70% by mass and most preferably at least 80% by mass of the active GLP ingredient and optional insulin ingredient of the respective ingredients of the metered dose.

In another aspect of the invention at least one excipient is in a formulation where the MMAD of the particles is 10 μm or more, such that the at least one excipient acts as a carrier for the finely divided particles of the at least one active GLP agent of the metered dose. Besides diluting the potency of the active GLP ingredient(s), excipients contribute to acceptable metering and dose forming properties of the powder mixture. When the metered dose is delivered to a user by means of a dry powder inhaler device (DPI), almost all of the excipient particle mass is deposited in the mouth and upper airways, because the AD of excipient particles are generally too big to follow the inspiration air into the lung. Therefore, excipients acting as carriers and/or diluents are selected inter alia with a view to being harmless when deposited in these areas.

Suitable carrier or diluent excipients for inclusion in a GLP formulation are to be found among the groups of monosaccarides, disaccarides, oligo- and polysaccarides, polylactides, polyalcohols, polymers, salts or mixtures from these groups, e.g. glucose, arabinose, lactose, lactose monohydrate, lactose anhydrous [i.e., no crystalline water present in lactose molecule], saccharose, maltose, dextrane, sorbitol, mannitol, xylitol, sodium chloride, calcium carbonate. A particular excipient is lactose.

In our experience many dry powder peptides are sensitive to moisture. Thus, the moisture properties of any proposed excipient must be checked before it is chosen to be included in a formulation comprising GLP and/or insulin, regardless of the intended function of the proposed excipient. If an excipient gives off much water, after dose forming, it will negatively affect the active ingredients in the dose, such that the FPD deteriorates rapidly after dose forming. Therefore, excipients are to be selected among acceptable excipients, which have good moisture properties in the sense that the excipient will not adversely affect the FPD of the active ingredients for the shelf life of the product, regardless of normal changes in ambient conditions during transportation and storage. Suitable “dry” excipients are to be found in the above-mentioned groups. In a particular embodiment of a GLP dose, optionally also comprising insulin, lactose is selected as the preferred dry excipient and preferably lactose monohydrate. A reason for selecting lactose as excipient, is its inherent property of having a low and constant water sorption isotherm. Excipients having a similar or lower sorption isotherm can also be considered for use, provided other required qualities are met.

The dose size depends on the type of disorder and the selected GLP agent for adequate therapy, but naturally age, weight, gender and severity of the medical condition of the subject undergoing therapy are important factors. According to the present invention, a delivered fine particle dose (FPD) of the active ingredient administered by inhalation herein is not limited, and may generally be in a range from 10 μg to 25 mg. Normally, of course, a physician prescribes a proper dose size. Depending on the potency of the active substance, such as GLP and human insulin agents, the active dose mass is optionally diluted by adding a pharmacologically acceptable excipient to the formulation to suit a particular method of dose forming and to achieve a pre-metered dose in the inhaler, preferably exceeding 100 μg. Besides acting as a diluent, the excipient may optionally be selected to give desired electrical qualities to the powder mixture constituting the drug. A method for preparing a powder or powder mixture to bring about suitable electrostatic properties of the prepared powder to make the powder apt for a filling process is described in our U.S. Pat. No. 6,696,090, which is hereby incorporated in this document in its entirety by reference.

Further, the correct metered dose loaded into an inhaler for administration must be adjusted for predicted losses such as retention and fine particle fraction (FPF) of the inhaled dose. A practical lower limit for volumetric dose forming is in a range 0.5 to 1 mg. Doses smaller than an order of 1 mg are difficult to produce while maintaining a low relative standard deviation between doses of the order of at least 5%. Typically, though, dose masses for inhalation are in a range from 1 to 50 mg.

Ambient conditions during dose forming, metering and container sealing should be closely controlled. The ambient temperature is preferably limited to 25° C. maximum and relative humidity preferably limited to 15% Rh maximum, although some drug formulations must be filled in very dry conditions of only a few percent relative humidity. As already mentioned in the foregoing it is very important to control the electric properties of the powder and thereby controlling the use of electric charging and discharging of particles, regardless of which method of dose forming is to be used. Fine powders pick up static electric charges extremely easily, which can be advantageously used in dose forming, if the charging and discharging is under proper control.

“High barrier seal” means a dry packaging construction or material or combinations of materials. A high barrier seal is wherein it represents a high barrier against moisture and that the seal itself is ‘dry’, i.e. it cannot give off measurable amounts of water to the load of powder. A high barrier seal may for instance be made up of one or more layers of materials, i.e. technical polymers, aluminum or other metals, glass, silicon oxides etc that together constitutes the high barrier seal. If the high barrier seal is a foil, a 50 μm PCTFE/PVC pharmaceutical foil is the minimum required high barrier foil if a two-week in-use stability for a moisture sensitive medicament shall be achieved. For longer in-use stabilities metal foils like aluminum foils from Alcan Singen can be used.

The medical product disclosed comprises a dose container as primary package, which may be a “high barrier seal container”. The disclosed dose container is a mechanical construction made to harbor and enclose a dose of e.g. GLP or insulin or a dose combination or a mixture thereof, which may be sensitive to humidity. The design of the dose container and the materials used must be adequate for the drug considering the sensitivity to humidity and the specified in-use time for the container as primary package. A sealed dose container can be made up of one or more layers of materials, i.e. technical polymers, aluminum or other metals, glass, silicon oxides etc and may exist in many different shapes, e.g. completely or partly spherical, cylindrical, box-like etc. However, the volume of the container is preferably not bigger than necessary for loading and enclosing a metered dose or dose combination, thereby minimizing the amount of moisture enclosed in the atmosphere. Another requirement is that the container is designed to facilitate opening thereof, preferably in a way that makes the enclosed dose accessible for direct release, aerosolization and entrainment of the powder in inspiration air during an inhalation. The time the dose is exposed to ambient air is thereby minimized. A high barrier seal container is built using high barrier seals constituting the enclosing, i.e. walls of the container.

The sealed, dry container of the present invention that is directly loaded with a GLP dose may be in the form of a blister and it may e.g. comprise a flat dose bed or a formed cavity in aluminum foil or a molded cavity in a polymer material, using a seal foil against ingress of moisture, e.g. of plastic or aluminum or a combination of aluminum and polymer materials. The sealed, dry, container may form a part of an inhaler device or it may form a part of a separate item intended for insertion into an inhaler device for administration of pre-metered doses. A particular embodiment of a sealed high barrier container used in an adapted DPI has the following data:

Container internal volume: 100 mm³

Effective diffusion area: 46 mm²

Diffusion constant: 0.044 g/m² for 24 hours at 23° C. and differential Rh=50% Rh

In a further aspect of the present invention the medical product comprises at least one GLP agent and at least one insulin agent in a combined metered dose, optionally including at least one biologically acceptable excipient, loaded and sealed into a dose container. A GLP dosage and an insulin dosage, which together constitute a combined dose, may be sharing the same dose container or the dosages may be separated into separate dose containers. Methods of producing the combined dose are known in the art and include spray-drying, lyophilizing, vacuum drying, open drying, jet milling and mixing. Each ingredient may be produced as separate formulations or may be introduced into a selected process producing a combined formulation of the ingredients, if safe with regard to chemical and biological stability and toxicology. It is further possible, according to the disclosure herein, to make the resulting formulation(s) as powder, optionally powder inter-mixtures, of finely divided particles, or large-sized porous particles. The sealed dose container of the medical product is thus protecting the combined dose from ingress of moisture and other foreign matter, thereby preserving the FPD of the combined peptide medicament for the specified in-use time period. Deterioration of the FPD is further protected by enclosing only an insignificant quantity of moisture inside the container together with the dose by keeping the humidity in the atmosphere during dose metering and forming to a sufficiently low level, and optionally by choosing the biologically acceptable excipient with as low sorption coefficient as possible. For instance, the humidity in the atmosphere where the powder is handled immediately prior to metering and forming should be kept below 15% Rh and preferably below 10% Rh, more preferably below 5% Rh and most preferably below 1% Rh. The disclosed medical product warrants that the quality of the delivered dose is high and intact over the full shelf life period and the in-use period of the product.

In FIGS., 4, 5, 6 and 7 reference numbers 11-42 of the drawings same numbers indicate like elements throughout the different embodiments of the medical product, presented here as non-limiting examples.

As used herein, the phrases “selected from the group consisting of,” “chosen from,” and the like include mixtures of the specified materials. All references, patents, applications, tests, standards, documents, publications, brochures, texts, articles, instructions, etc. mentioned herein are incorporated herein by reference. Where a numerical limit or range is stated, the endpoints are included. Also, all values and sub-ranges within a numerical limit or range are specifically included as if explicitly written out.

In the context of this document all references to ratios, including ratios given as percentage numbers, are related to mass, if not explicitly said to be otherwise.

1. A medical product comprising a sealed dose container, said container comprising therein:
a metered, dry powder medicament dose of at least one active, glucagon-like peptide (GLP) agent;
the medicament dose optionally further comprising an active insulin agent, the insulin agent comprising at least one peptide of recombinant, human insulin or insulin analogue;
the medicament dose optionally further comprising at least one biologically acceptable excipient;
the medical product being adapted for a pulmonary delivery of the medicament dose by inhalation from a dry powder inhaler, and
the medicament dose of the medical product being arranged to be aerosolized and entrained into inspiration air directly from the container when opened by the inhaler, the medicament dose being further arranged to be aerosolized exclusively by the inhalation power of a user for the pulmonary delivery, whereby more than 50% by mass of each of the respective active agents of the medicament dose leaves the inhaler as a fine particle dose (FPD).

2. The medical product according to claim 1, wherein the medicament dose comprises the active insulin agent.

3. The medical product according to claim 2, wherein the active agents of the medicament dose are provided as an inter-mixture in the container.

4. The medical product according to claim 2, wherein the active agents of the medicament dose are provided separately in the container, each active agent optionally further comprising at least one biologically acceptable excipient.

5. The medical product according to claim 2, wherein the medical product comprises an amount of insulin agent in a range from 100 μg to 25 mg in the medicament dose.

6. The medical product according to claim 1, wherein the GLP agent is selected from a GLP sequence or a pharmaceutically acceptable analogue or derivate thereof.

7. The medical product according to claim 1, wherein the GLP agent comprises. GLP-1 or a pharmaceutically acceptable analogue or derivate thereof.

8. The medical product according to claim 1, wherein the GLP agent comprises GLP-2 or a pharmaceutically acceptable analogue or derivate thereof.

9. The medical product according to claim 1, wherein the prolonged pulmonary delivery of a dose of the medical product takes place in a period of not less than 0.1 s and not more than 5 s.

10. The medical product according to claim 1, wherein the required inhalation power for de-aggregating and aerosolizing a dose of the medical product is not less than 2 kPa and not more than 6 kPa of air pressure resulting in an inspiration air flow of not less than 20 l/min and not more than 60 l/min.

11. The medical product according to claim 1, wherein more than 60% by mass of the active agent or each of the respective active agents of the medicament dose leaves the inhaler as a FPD.

12. The medical product according to claim 1, wherein a total mass of the GLP agent in the medicament dose of the medical product is in a range from 10 μg to 25 mg of a total dose mass in a range from 1 mg to 50 mg.

13. The medical product according to claim 1, wherein the dry powder medicament dose has a mass median aerodynamic diameter in a range from 1 to 3 μm.

14. The medical product according to claim 1, wherein the at least one, optional dry excipient of the medical product is present and comprises particles having a diameter of 25 μm or more in an amount of more than 40% by mass based on total mass of excipient, and the at least one, optional dry excipient further comprises an excipient selected from a group consisting of monosaccarides, disaccarides, polylactides, oligo- and polysaccarides, polyalcohols, polymers, salts or mixtures thereof.

15. The medical product according to claim 1, wherein the container of the medical product constitutes a high barrier seal container protecting the medicament dose from ingress of moisture, whereby the integrity of the medicament dose is fully protected for the shelf-life of the medical product.

16. A dry powder inhaler comprising a medical product according to claim 1.

17. A method of producing a medical product, said method comprising the steps of
providing a dry powder medicament dose of at least one active, glucagon-like peptide (GLP) agent, optionally an active insulin agent, the insulin agent comprising at least one peptide of recombinant, human insulin or insulin analogue, and optionally at least one biologically acceptable excipient in a dose container; and
sealing the dose container,
wherein the medical product is adapted for a pulmonary delivery of the medicament dose by inhalation from a dry powder inhaler, and
the medicament dose of the medical product is adapted to be aerosolized and entrained into inspiration air exclusively by the inhalation power of a user directly from the container when opened by the inhaler.

18. The method according to claim 17, wherein the medicament dose comprises the active insulin agent.

19. A method of emitting a dry powder medicament dose of a medical product according to claim 1 comprising the steps of:
arranging the medical product in a dry powder inhaler in such a way that the medicament dose of the medical product is aerosolized and entrained into inspiration air directly from the container when opened by the inhaler; and
applying a suction effort to the inhaler, whereby the medicament dose is aerosolized exclusively by the inhalation power provided by the suction effort for a prolonged pulmonary delivery, whereby more than 50% by mass of each of the respective active agents of the medicament dose leaves the inhaler as a fine particle dose, FPD.

20. The method according to claim 19 comprising the further steps of
providing the suction effort by machine operated means, and
mimicking pulmonary delivery by a mechanical in-vitro means.
